(19) United States
(12) Patent Application Publication
(10) Pub. No.: US 2024/0221939 A1
(43) Pub. Date: Jul. 4, 2024

(54) SYSTEM AND METHOD FOR AUTOMATED DISCOVERY OF TIME SERIES TRENDS WITHOUT IMPUTATION

(71) Applicant: KONINKLIJKE PHILIPS N.V., EINDHOVEN (NL)

(72) Inventor: JUNZI DONG, CAMBRIDGE, MA (US)

(21) Appl. No.: 18/289,127

(22) PCT Filed: May 4, 2022
(86) PCT No.: PCT/EP2022/062019

§ 371 Date: Nov. 1, 2023

Related U.S. Application Data
(60) Provisional application No. 63/184,893, filed on May 6, 2021.

(51) Int. Cl. G16H 50/20; G16H 40/20

(52) U.S. Cl. CPC G16H 50/20; G16H 40/20

(57) ABSTRACT

A method (100) for automated prediction of a clinical state, comprising: receiving (120) a set of parameter definitions for a clinical state, the set of parameter definitions comprising a definition for a deviation value, a definition for a deviation time, a definition for a value threshold, and a definition for a value time; receiving (130) a plurality of measurements for at least one feature for a patient, the plurality of measurements taken over a span of time; identifying (140), within the plurality of measurements for at least one feature for a patient, a deviation and/or an abnormality predicting the clinical state, comprising: predicting (150), based upon identification of a deviation and/or abnormality, that the patient is susceptible to or experiencing the clinical state; and providing (160), via a user interface of the event monitoring system, an alert that the patient is susceptible to or experiencing the clinical state.

### FIELD OF THE INVENTION

The present disclosure is directed generally to systems and methods for automated prediction of a clinical state using an event monitoring system.

### BACKGROUND

Clinical time series data are known to be very informative for patient conditions. Clinicians informally track temporal trends of vitals to monitor patients, including one or both of two types of trends: deviations over time and abnormal values over time. For example, a deviation such as a rapid heart rate increase may indicate pain or agitation, and an abnormal value such as a sustained low blood pressure may indicate onset of shock.

However, quantitative definition of trends over time is not well defined for most patient conditions. Traditionally, any tracking of trends is based on a single clinician's experience and mental model. For example, a clinician may have a hard time quantifying ‘rapid heart rate increase’ in the first example (e.g., is a 50 BPM increase over 10 min a rapid heart rate increase?), or ‘sustained low blood pressure’ in the second example (e.g., is a systolic BP under 70 mmHg for 20 min an abnormal value?).

Further, one of the challenges of working with clinical time series data is that they are irregularly sampled. Many computational models and techniques require regular samples. To overcome this, data imputation is typically performed to interpolate between sample times. However, this changes the data and can create artifacts that cause inaccuracies in predictions.

Existing cutting-edge methods for pattern mining in clinical time series are mainly recurrent neural networks (RNN). However, RNNs are notorious for their lack of interpretability. Any patterns found are hidden in the numerous neural network coefficients, which remain difficult for clinicians to understand. Additional, RNNs are more computationally expensive than the implementation of this invention, which only requires two to four threshold parameters per time series.

### SUMMARY OF THE INVENTION

Accordingly, there is a continued need in the art for systems and methods that automatically define quantitative temporal trends from clinical data and enable automated monitoring of patients.

The present disclosure is directed to inventive methods and systems for the automated prediction of a clinical state using an event monitoring system and a plurality of patient features. Various embodiments and implementations herein are directed to a system that automatically identifies quantitative trends from big-data time series without imputation. The system identifies deviations and cutoffs that support clinicians' analysis and are straightforward for clinicians to understand and trust. The system is programmed or otherwise provided with a set of parameter definitions for a clinical state, comprising a definition for a deviation value, a definition for a deviation time, a definition for a value threshold, and a definition for a value time. The system receives, for one or more patients, a plurality of measurements taken over a span of time. The system then identifies within the plurality of measurements for at least one feature for a patient, a deviation and/or an abnormality predicting the clinical state. To identify a deviation, the system compares the received plurality of measurements for the patient to the defined deviation value and the defined deviation time, and identifies a deviation if the received plurality of measurements taken over the span of time for the patient is above the defined deviation value and below the defined deviation time. To identify an abnormality, the system compares the received plurality of measurements for the patient to the defined value threshold and the defined value time, and identifies an abnormality if the received plurality of measurements taken over the span of time for the patient is above the defined value threshold and below the defined value time. The system predicts that the patient is susceptible to or experiencing the clinical state based upon identification of a deviation and/or abnormality. The system provides, via a user interface, an alert that the patient is susceptible to or experiencing the clinical state.

Generally, in one aspect, a method for automated prediction of a clinical state using an event monitoring system is provided. The method includes: receiving a set of parameter definitions for a clinical state, the set of parameter definitions comprising a definition for a deviation value, a definition for a deviation time, a definition for a value threshold, and a definition for a value time; receiving a plurality of measurements for at least one feature for a patient, the plurality of measurements taken over a span of time; identifying, within the plurality of measurements for at least one feature for a patient, a deviation and/or an abnormality predicting the clinical state, comprising: (i) comparing, using a batch deviation detection module of the event monitoring system, the received plurality of measurements for the patient to the defined deviation value and the defined deviation time; and (ii) identifying, based on the comparing step, a deviation if the received plurality of measurements taken over the span of time for the patient is above the defined deviation value and below the defined deviation time; and/or (iii) comparing, using the batch deviation detection module of the event monitoring system, the received plurality of measurements for the patient to the defined value threshold and the defined value time; and (iv) identifying, based on the comparing step, an abnormality if the received plurality of measurements taken over the span of time for the patient is above the defined value threshold and below the defined value time; predicting, based upon identification of a deviation and/or abnormality, that the patient is susceptible to or experiencing the clinical state; and providing, via a user interface of the event monitoring system, an alert that the patient is susceptible to or experiencing the clinical state.

According to an embodiment, the plurality of measurements comprise measurements for a plurality of patients, and the method further comprises the step of organizing the plurality of measurements with individual patients within the plurality of patients.

According to an embodiment, the method further includes the step of extracting one or more features from the received plurality of measurements.

According to an embodiment, the feature is a vital sign, a lab result, and/or a clinical score.

According to an embodiment, the method further includes the steps of: receiving training data comprising medical information and clinical state information for a plurality of patients; extracting a plurality of features from the received training data; and training the batch deviation detection module.

According to an embodiment, the event monitoring system comprises a clinical care system configured to monitor multiple life signs for a patient, and wherein the user interface of the event monitoring system comprises a display of the monitored multiple life signs for the patient, and wherein the alert that the patient is susceptible to or experiencing the clinical state comprises a textual alert on the display.

According to an embodiment, the event monitoring system is utilized to determine a set of parameter definitions for a clinical state during training of the event monitoring system.

According to an aspect is an event monitoring system. The system includes: a set of parameters definitions for a clinical state, the set of parameter definitions comprising a definition for a deviation value, a definition for a deviation time, a definition for a value threshold, and a definition for a value time; a plurality of measurements for at least one feature for a patient, the plurality of measurements taken over a span of time; a trained batch deviation detection module; a processor configured to identify, within the plurality of measurements for at least one feature for a patient, a deviation and/or an abnormality predicting the clinical state, comprising: (i) comparing, using a batch deviation detection module of the event monitoring system, the received plurality of measurements for the patient to the defined deviation value and the defined deviation time; (ii) identifying, based on the comparing step, a deviation if the received plurality of measurements taken over the span of time for the patient is above the defined deviation value and below the defined deviation time; (iii) comparing, using the batch deviation detection module of the event monitoring system, the received plurality of measurements for the patient to the defined value threshold and the defined value time; and (iv) identifying, based on the comparing step, an abnormality if the received plurality of measurements taken over the span of time for the patient is above the defined value threshold and below the defined value time; the processor is further configured to predict, based upon identification of a deviation and/or abnormality, that the patient is susceptible to or experiencing the clinical state; and a user interface configured to provide an alert that the patient is susceptible to or experiencing the clinical state.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects of the present invention discussed herein. The terms “program” or “computer program” are used herein in a generic sense to refer to any type of computer code (e.g., software or microcode) that can be employed to program one or more processors or controllers.

In one network implementation, one or more devices coupled to a network may serve as a controller for one or more other devices coupled to the network (e.g., in a master/slave relationship). In another implementation, a networked environment may include one or more dedicated controllers that are configured to control one or more of the devices coupled to the network. Generally, multiple devices coupled to the network each may have access to data that is present on the communications medium or media; however, a given device may be “addressable” in that it is configured to selectively exchange data with (i.e., receive data from and/or transmit data to) the network, based, for example, on one or more particular identifiers (e.g., “addresses”) assigned to it.

The term “network” as used herein refers to any interconnection of two or more devices (including controllers or processors) that facilitates the transport of information (e.g. for device control, data storage, data exchange, etc.) between any two or more devices and/or among multiple devices coupled to the network. As should be readily appreciated, various implementations of networks suitable for interconnecting multiple devices may include any of a variety of network topologies and employ any of a variety of communication protocols. Additionally, in various networks according to the present disclosure, any one connection between two devices may represent a dedicated connection between the two systems, or alternatively a non-dedicated connection. In addition to carrying information intended for the two devices, such a non-dedicated connection may carry information not necessarily intended for either of the two devices (e.g., an open network connection). Furthermore, it should be readily appreciated that various networks of devices as discussed herein may employ one or more wireless, wire/cable, and/or fiber optic links to facilitate information transport throughout the network.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 is a flowchart of a method for identifying a deviation and/or an abnormality within a plurality of measurements taken over time for a patient, in accordance with an embodiment.

FIG. 2 is flowchart of a method for identifying a deviation and/or an abnormality within a plurality of measurements taken over time for a patient, in accordance with an embodiment.

FIG. 3 is a flowchart of a method for patient event detection, in accordance with an embodiment.

FIG. 4 is a flowchart of a method for patient event detection, in accordance with an embodiment.

FIG. 5 is flowchart of a method for training a classifier, in accordance with an embodiment.

FIG. 6 is a schematic representation of a system for identifying a deviation and/or an abnormality within a plurality of measurements taken over time for a patient, in accordance with an embodiment.

FIG. 7 is a graph of an example AKI patient, in accordance with an embodiment.

FIG. 8 is a graph of an example non-AKI patient, in accordance with an embodiment.

FIG. 9 is an example of an event detection system, in accordance with an embodiment.

### DETAILED DESCRIPTION OF EMBODIMENTS

The present disclosure describes various embodiments of a patient monitoring system configured to automatically identify a deviation and/or an abnormality within a plurality of measurements taken over time for a patient. More generally, Applicant has recognized that it would be beneficial to provide a patient monitoring system that can automatically predict a clinical state for a patient. In view of the foregoing, various embodiments and implementations are directed to a monitoring system that is programmed or otherwise provided with a set of parameter definitions for a clinical state, comprising a definition for a deviation value, a definition for a deviation time, a definition for a value threshold, and a definition for a value time. The system receives, for one or more patients, a plurality of measurements taken over a span of time. The system then identifies within the plurality of measurements for at least one feature for a patient, a deviation and/or an abnormality predicting the clinical state. To identify a deviation, the system compares the received plurality of measurements for the patient to the defined deviation value and the defined deviation time, and identifies a deviation if the received plurality of measurements taken over the span of time for the patient is above the defined deviation value and below the defined deviation time. To identify an abnormality, the system compares the received plurality of measurements for the patient to the defined value threshold and the defined value time, and identifies an abnormality if the received plurality of measurements taken over the span of time for the patient is above the defined value threshold and below the defined value time. The system predicts that the patient is susceptible to or experiencing the clinical state based upon identification of a deviation and/or abnormality. The system provides, via a user interface, an alert that the patient is susceptible to or experiencing the clinical state.

Accordingly, the patient monitoring system comprises a method for detecting patient states of interest such as shock, hemodynamic instability, and many others by identifying temporal events. According to an embodiment, a temporal event occurs when a patient time series (such as blood pressure measured over an hour, as one example) matches a quantitatively defined trend over time. According to an embodiment, a clinical state is a patient condition that is being predicted by monitoring the time series trend of the patient. For example, a clinical state may be a patient developing shock, experiencing hemodynamic instability, and many other conditions.

The embodiments and implementations disclosed or otherwise envisioned herein can be utilized with any monitoring system, including but not limited to medical monitoring devices or systems. For example, one application of the embodiments and implementations herein is to improve medical monitoring systems such as, e.g., a Philips Patient Monitoring system (manufactured by Koninklijke Philips, N.V.). However, the disclosure is not limited to a medical monitoring device or system and thus the disclosure and embodiments disclosed herein can encompass any monitoring device or system.

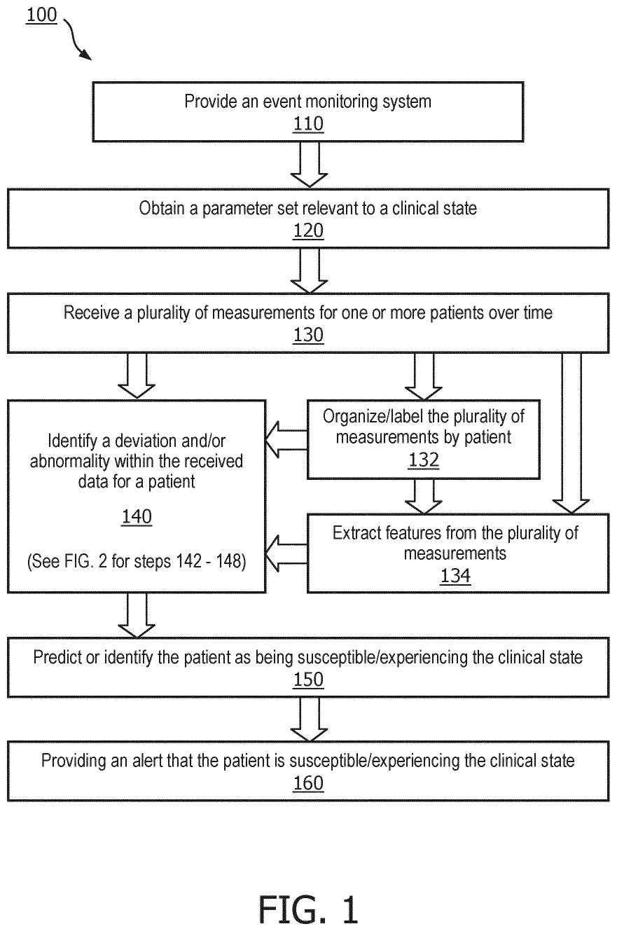

Referring to FIG. 1 is a flowchart of a method 100 for automatically predicting a clinical state using an event monitoring system. At step 110 of the method, an event monitoring system is provided. The event monitoring system can be any of the systems described or otherwise envisioned herein. According to one embodiment, the event monitoring system can be a permanent installation or can be a handheld or other portable device, such as a personal device like a smartphone, tablet, or other device. Alternatively, the event monitoring system can be a medical monitoring device or system, or a component of a medical monitoring device or system.

At step 120 of the method, the event monitoring system receives, accesses, is programmed or set with, or otherwise obtains a parameter set for or otherwise relevant to a clinical state. As described or otherwise envisioned herein, the event monitoring system analyzes patient data, which includes data such as patient measurements, to identify deviations and/or abnormalities over time that indicate a possibility or probability of a clinical state. In order to identify a deviation and/or an abnormality in patient data, the event monitoring system requires a parameter set with which to analyze the patient data. According to an embodiment, the event monitoring system can be manufactured or otherwise programmed with the parameter set for one or more clinical states. According to an embodiment, the event monitoring system comprises a parameter set for numerous different clinical states.

According to an embodiment, the parameter set received or obtained by the event monitoring system comprises one or more of the following parameters: (i) a definition for a deviation value; (ii) a definition for a deviation time; (iii) a definition for a value threshold (as used herein, the term ‘value threshold,’ also called ‘v_value,’ is a threshold for a clinical state parameter); and (iv) a definition for a value time. According to an embodiment, the parameter set received or obtained by the event monitoring system also comprises one or more of: (i) a deviation direction; and (ii) a value direction. The deviation/value direction parameters govern the direction of time series data change, and they can be either + or −, indicating increases or decreases. This enables the model to learn which direction best predicts the physiological condition of interest. These parameters are further discussed and described herein.

There are an unlimited number of example clinical states and associated parameters. A few non-limiting examples are discussed herein. For example, the clinical state may be shock. Parameters that can be utilized for shock include blood pressure such as that measured by invasive (arterial or central line) and blood pressure cuff measured values, heart rate such as that measured by ECG, and/or laboratory values such as hemoglobin, hematocrit, lactate, calcium, and other measurements. As another example, the clinical state may be acute respiratory distress (ARDS). Parameters that can be utilized for ARDS include heart rate such as that measured by ECG, SpO₂ such as that measured by oximetry, and/or ventilation parameters including FiO₂ and PEEP. As another example, the clinical state may be infection or sepsis. Parameters that can be utilized for infection or sepsis include heart rate such as that measured by ECG, temperature values such as that measured by probes or spot checks, and/or laboratory values such as white blood cell count.

According to an embodiment, the parameter set received or obtained by the event monitoring system is stored in a memory or other data storage structure of the event monitoring system, for retrieval during operation of the system. For example, the event monitoring system can be periodically updated with new or updated parameter sets for one or more clinical states. Alternatively, the parameters can be stored or otherwise hosted at a remote location. Thus, the event monitoring system can request the most up-to-date parameters for a clinical state when the system will be monitoring for that clinical state. Accordingly, the event monitoring system can be designed or configured to communicate with a remote server, database, or other remote element in order to request and retrieve or otherwise obtain the parameter set. For example, since the event monitoring system can be designed to monitor a wide variety of clinical states, and thus it can access the remote element to request the most up-to-date parameters for a requested clinical state.

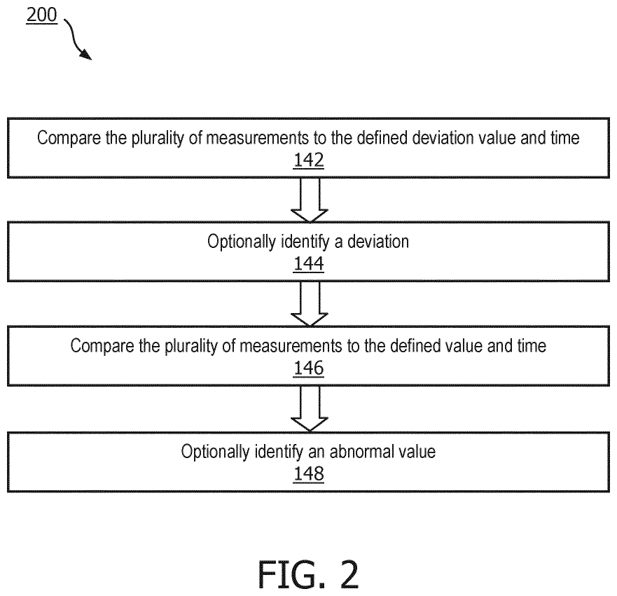

According to an embodiment, one or more of the parameter definitions are determined or defined during training of the event monitoring system. Training of the event monitoring system is described below in regard to FIG. 2.

At step 130 of the method, the event monitoring system receives, accesses, is programmed or set with, or otherwise obtains a plurality of measurements for a patient. These plurality of measurements are taken over a span of time. According to an embodiment, the plurality of measurements are provided to the event monitoring system in real-time or in one or more batches of data. The event monitoring system may continually or periodically request measurements for one or more patients. Accordingly, the measurements may be provided in real-time or retrieved from a database of patient measurements. For example, the event monitoring system may be in communication with a local or remote electronic medical record database and can be programmed or otherwise designed to receive, request, or otherwise access measurements or other records for a patient.

According to an embodiment, a measurement for a patient comprises any measurement that might be relevant to a clinical state. As just a few examples, the measurement can include patient demographics such as age, gender, and more; diagnosis or medication condition such as cardiac disease, psychological disorders, chronic obstructive pulmonary disease, and more; physiologic vital signs such as heart rate, blood pressure, respiratory rate, oxygen saturation, and more; telemetry alarm related data such as arrhythmia-related alarms (ventricular tachycardia, ventricular bradycardia, atrial fibrillation, asystole, and more); physiologic measurements such as heart rate, respiratory rate, apnea, SpO₂, invasive arterial pressure, noninvasive blood pressure, and more; and/or technical alarms such as artifact, ECG leads fail, respiratory leads fail, blood pressure sensor fail, and more. Many other types of input data are possible.

According to an embodiment, the plurality of measurements for the patient are made at irregular intervals during the time span. For example, a patient's blood pressure may be taken at 1:15 PM, 1:58 PM, 3:05 PM, and 4:30 PM. The intervals (35 minutes, 67 minutes, and 85 minutes) are highly irregular. However, because the event monitoring system has been trained to process measurements taken at irregular intervals, the system is still able to analyze the data to identify a deviation and/or abnormality. Indeed, the ability of the event monitoring system to analyze patient measurements taken at irregular intervals during the time span is just one of the many advantages provides by the novel event monitoring system.

At optional step 132 of the method, the event monitoring system analyzes the data received in step 130, and organizes the data to separate it into individual patients, or to otherwise label or identify the data by individual patient. Data points in the received measurements will be associated with a particular patient using a patient identifier or other identification. Accordingly, the patient identifier can be utilized to organize the plurality of measurements into measurements for person i, person i+1, and so on. Alternatively, the system may be designed or otherwise configured to only monitor data for a single patient. Accordingly, the system is configured to identify only measurements relevant to the single patient using the patient identifier. In this case, the system can pull only relevant measurements out of the received data. The organized measurements may be appropriately labeled or tagged or provided with an identifier in memory, or the event monitoring system can store organized or separated measurements in memory by individual patient.

At optional step 134 of the method, the event monitoring system extracts one or more features from the plurality of measurements for a patient. This can be accomplished by a variety of embodiments for feature identification, extraction, and/or processing, including any method for extracting features from a dataset. The outcome of a feature processing step or module of the event monitoring system is a set of features related to measurements about the patient.

At step 140 of the method, the event monitoring system identifies, within the plurality of measurements for at least one feature for a patient, a deviation and/or an abnormality that predicts or potentially predicts a clinical state. Deviations and/or abnormalities that predict the possibility or probability of a clinical state can be identified in many different ways. According to an embodiment, the event monitoring system described or otherwise envisioned herein identifies deviations and/or abnormalities as described in the following steps, although other methods are possible.

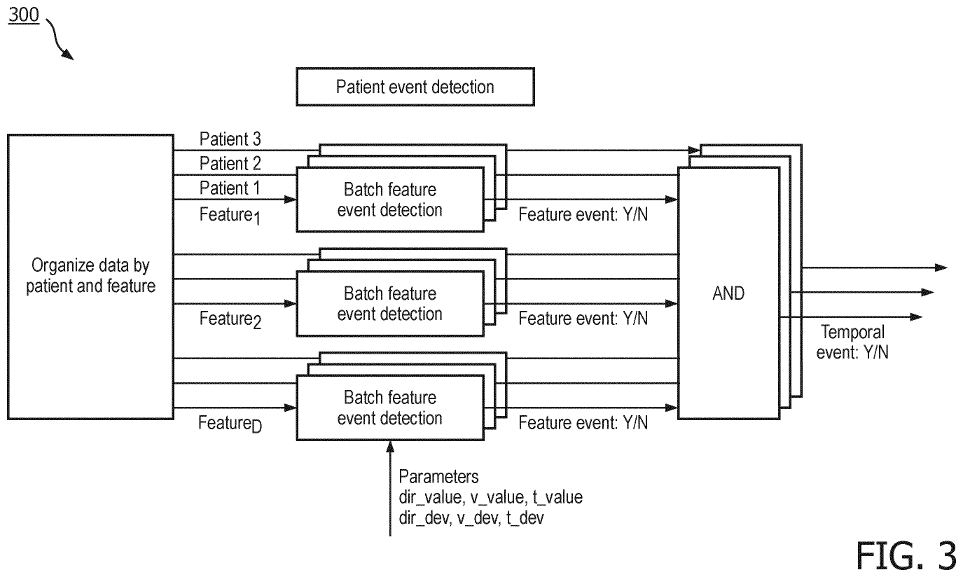

Referring to the flowchart in FIG. 2 for identifying deviations and/or abnormalities, at step 142 of the method, the event monitoring system compares, using a batch deviation detection module of the event monitoring system, the received plurality of measurements for the patient to the defined deviation value and the defined deviation time. Referring to FIG. 3, for example, is a flowchart of the method, in which features a large set of time-series data for multiple patients and multiple features first organized by patients. The original time series data for all features of each patient are passed through the batch feature event detection module of the system, which computes whether a specific feature matches the feature event criteria. The batch feature event detection module looks for a temporal trend, including deviation over time and/or an abnormal value over time, using the parameter set for a clinical state. In this step, the batch feature event detection module looks for a deviation over time using one or more of the defined deviation value, the defined deviation time, and/or the defined deviation direction.

At step 144 of the method, the event monitoring system identifies, based on the comparing step, a deviation if some or all of the received plurality of measurements taken over the span of time for the patient is above the defined deviation value and below the defined deviation time.

At step 146 of the method, the event monitoring system compares, using the batch deviation detection module of the event monitoring system, the received plurality of measurements for the patient to the defined value threshold and the defined value time. Referring to FIG. 3, the original time series data for all features of each patient are passed through the batch feature event detection module of the system, which computes whether a specific feature matches the feature event criteria. The batch feature event detection module looks for a temporal trend, including deviation over time and/or an abnormal value over time, using the parameter set for a clinical state. In this step, the batch feature event detection module looks for an abnormal value over time using one or more of the defined value threshold, the defined value time, and/or the value deviation direction.

At step 148 of the method, the event monitoring system identifies, based on the comparing step, an abnormality if the received plurality of measurements taken over the span of time for the patient is above the defined value threshold and below the defined value time.

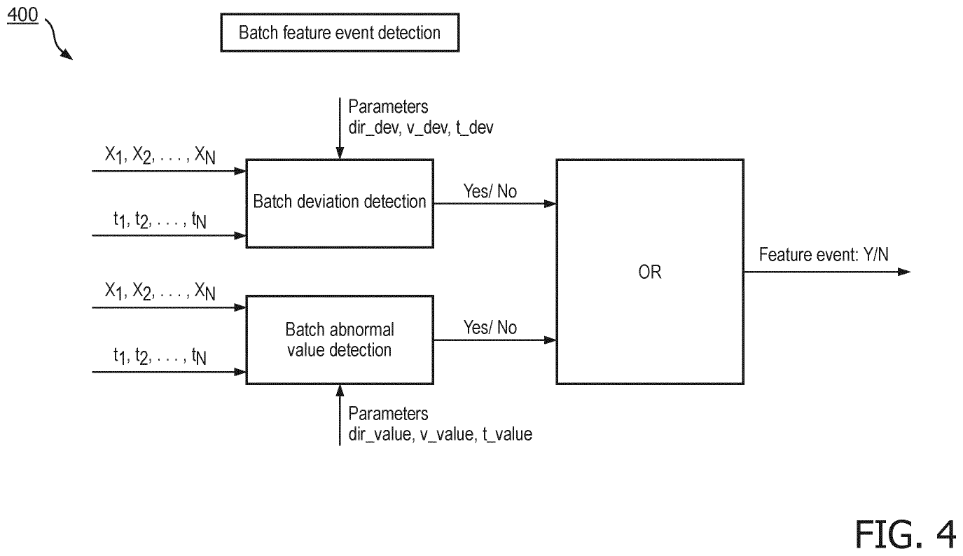

Referring to FIG. 4, in one embodiment, is a portion 300 of an event monitoring system. Features (x₁ . . . x_{N}) for a patient over time (t₁ . . . t_{N}) are fed into the batch feature event detection module of the system. According to an embodiment, the batch feature event detection module comprises a batch deviation detection module and a batch abnormal value detection module. The batch deviation detection module computes whether there is a significant deviation (increase or decrease) greater than [v_dev] in a short amount of time under [dt_dev], and the batch abnormal value detection module computes whether the value stays abnormal above or below a physiologically-critical threshold [v_value] in for a significant amount of time [dt_value].

Examples of computational formulations for the two modules—the batch deviation detection module and the batch abnormal value detection module—are shown in the pseudo-code below. This is provided only for illustration and is therefore a non-limiting example.

### Batch Abnormal Value Detection Pseudo-Code

```
  Input:
  - Sequences: t_seq, v_seq
  - Parameters: direction, t_dev, v_dev
  Output:
  - Boolean: feature_event
  if direction==‘less', then
      cross_thres = (v_seq <=v_dev) # Boolean sequence of when the threshold is crossed
  if direction==“greater’, then
      cross_thres = (v_seq >=v_dev) # Boolean sequence of when the threshold is crossed
  seq_diff = diff(seq) # discrete different
  mask_start = concatenate ([[v_seq[0]], seq_diff = = 1])
  t_start = mask_start * t_seq
  t_start = np.array([_for_in t_start if (not_ = =0)])# of segments where value begins to cross
threshold
  mask_end = concatenate([seq_diff = = −1,[v_seq[−1]])
  t_end = mask_end * t_seq
  t_end = np.array([_f_in t_end if (not_ = = 0)]) # time segements where value crosses normal where
value crosses threshold back to normal value
  dt =t_end − t_start
    Batch deviation detection pseudo-code
  Input:
  - Sequences: t_seq, v_seq
  - Parameters: direction, t_dev, v_dev
  Output:
  - Boolean: feature_event
  idx_pairs = list(combinations(range(len(t_seq)),2)) # get all combinations of samples in sequence
  n_pairs = len (idx pairs) # number of all combinations
  dv = [v_seq[idx_pairs[_][1]] − v_seq[idx_pairs[_][0]]) for_in range(n_pairs)] # compute all value
differences between samples
  dv = [t_seq[idx_pairs[_][1]] − t_seq[idx_pairs[_][0]]) for_in range(n_pairs)] # compute all time
differences between samples
  if direction = = ‘decrease’, then
      triggers = (dt>0 AND dt <= t_dev) AND dv <=v_dev #c check for decrease over v_dev in
under t_dev
  if direction = = ‘increase’, then
      triggers = (dt>0 AND dt <= t_dev) AND dv >=v_dev #c check for increase over v_dev in
under t_dev
  feature_event = any(triggers)
```

According to an embodiment, the temporal parameters (including but not limited to dir_value, t_value, v_value, dir_dev, t_dev, v_dev) can be found iteratively using most optimization techniques, including gradient descent and hyperopt. The batch processing of patient events enables the temporal parameters to be quickly iterated and optimized for.

According to an embodiment, the temporal parameters (including but not limited to dir_value, t_value, v_value, dir_dev, t_dev, v_dev) are initially set by a clinician or other entity within or near a predicted likely range for a clinical event. The parameters will likely then optimize as the training data is analyzed. Determined parameters can then be utilized by the event monitoring system.

At step 150 of the method, the event monitoring system predicts, based upon identification of a deviation and/or abnormality, that the patient is susceptible to or experiencing the clinical state. Referring to FIG. 3, a patient is labeled YES or Y—that is, the patient is susceptible to or experiencing the clinical state—if all feature events are satisfied. Clinically, a feature event occurring (Y) indicates that the patient satisfies the temporal trends a clinician is looking for, making that patient highly probably for developing the clinical state of interest. The feature events may comprise one feature or multiple features.

At step 160 of the method, the event monitoring system provides, via a user interface of the event monitoring system, an alert that the patient is susceptible to or experiencing the clinical state. There are many ways to display this information, and it may be displayed locally or remotely.

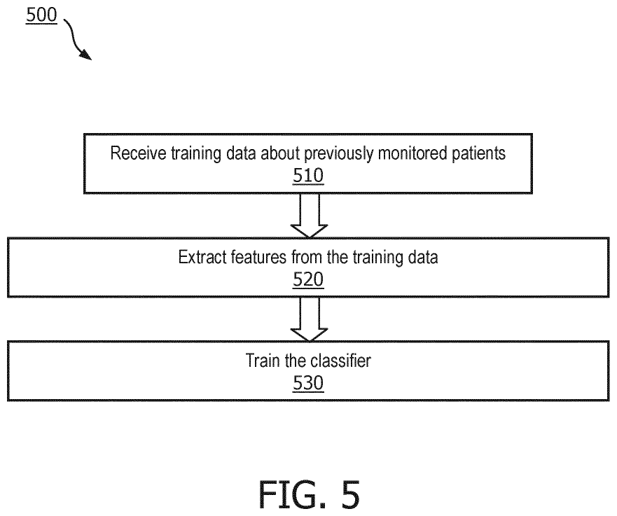

Referring to FIG. 5, according to an embodiment, is a flowchart of a method 500 for training an event monitoring system. According to an embodiment, the event monitoring system comprises a machine learning algorithm, also called a classifier, that has been trained to determine that a patient is susceptible to or experiencing a clinical state based on whether there is a significant deviation (increase or decrease) greater than [v_dev] in a short amount of time under [dt_dev], and/or whether the value stays abnormal above or below a physiologically-critical threshold [v_value] in for a significant amount of time [dt_value]. For example, the batch feature event detection module which can comprise a batch deviation detection module and a batch abnormal value detection module, can comprise the one or more machine learning algorithms. The machine learning algorithm of the event monitoring system can be trained using a dataset of historical data, the dataset comprising for each of a plurality of patients medical information about the patients, including development or non-development of a clinical state. Features are extracted from the historical patient information and utilized to train the machine learning algorithm of the event monitoring system.

According to an embodiment, the event monitoring system may be embodied in whole or in part within a device. For example, the entire event monitoring system may be embodied within a single device such as a handheld device, laptop, computer, or other single device. Alternatively, the event monitoring system may comprise a user interface that is transportable, such as a handheld device, mobile phone application, computer, or other transportable element that functions as a user interface to receive information. The device will communicate the information to the another, remote component of the event monitoring system for analysis. The result of the event monitoring system may then be communicated back to the transportable user interface.

At step 510 of the method, the event monitoring system receives input data comprising training data about a plurality of patients. The training data can comprise medical information about each of the patients, including but not limited to demographics, physiological measurements such as vital data, physical observations, and/or diagnosis, among many other types of medical information. As an example, the medical information can include detailed information on patient demographics such as age, gender, and more; diagnosis or medication condition such as cardiac disease, psychological disorders, chronic obstructive pulmonary disease, and more; physiologic vital signs such as heart rate, blood pressure, respiratory rate, oxygen saturation, and more; and/or physiologic data such as heart rate, respiratory rate, apnea, SpO₂, invasive arterial pressure, noninvasive blood pressure, and more. Many other types of medical information are possible.

The training data may also comprise an indication or information about whether the patient developed the clinical state for which the event monitoring system will be monitoring. For example, the training data may reveal that patient p went into shock at time t, as well as measurements, lab data, and/or other information taken for p over time. Additionally, the training data may reveal that patient p₊₁ did not go into shock at all, as well as measurements, lab data, and/or other information taken for p₊₁ over time.

This training data may be stored in and/or received from one or more databases. The database may be a local and/or remote database. For example, the event monitoring system may comprise a database of training data.

According to an embodiment, the event monitoring system may comprise a data pre-processor or similar component or algorithm configured to process the received training data. For example, the data pre-processor analyzes the training data to remove noise, bias, errors, and other potential issues. The data pre-processor may also analyze the input data to remove low quality data. Many other forms of data pre-processing or data point identification and/or extraction are possible.

At step 520 of the method, the system extracts patient features from the received training data. This can be accomplished by a variety of embodiments for feature identification, extraction, and/or processing, including any method for extracting features from a dataset. The outcome of a feature processing step or module of the event monitoring system is a set of patient features related to medical information and clinical states about a patient, which thus comprises a training data set that can be utilized to train the classifier.

At step 530 of the method, the system trains the machine learning algorithm, which will be the classifier utilized to analyze patient information as described or otherwise envisioned herein. The machine learning algorithm is trained using the extracted features according to known methods for training a machine learning algorithm. Following step 530, the event monitoring system comprises a trained classifier that can be utilized to classify patient status and provide a prediction of the patient's likelihood of being in or developing a clinical state. The trained classifier can be static such that it is trained once and is utilized for classifying. According to another embodiment, the trained classifier can be more dynamic such that it is updated or re-trained using subsequently available training data. The updating or re-training can be constant or can be periodic.

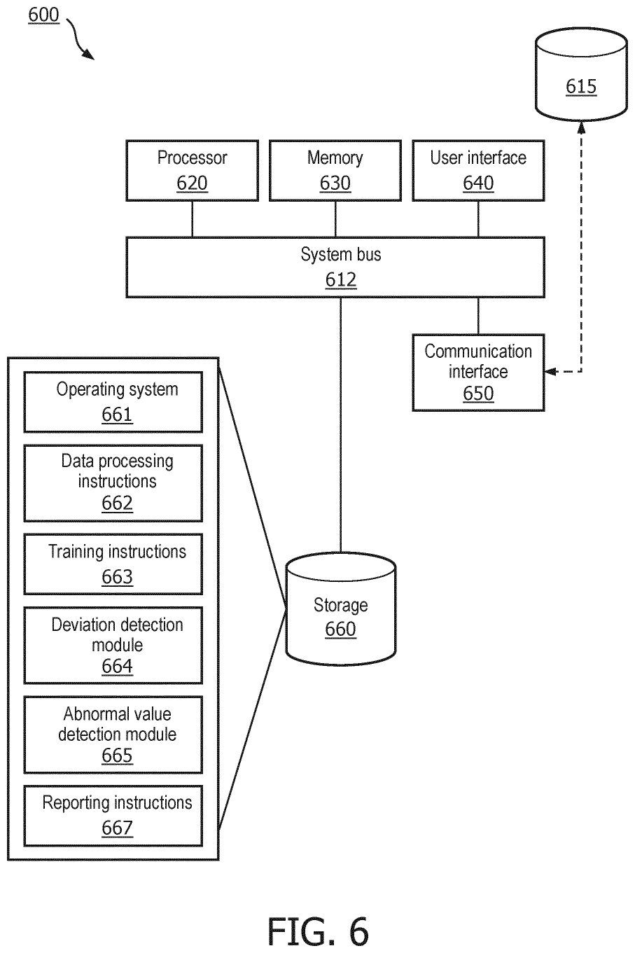

Referring to FIG. 6, in one embodiment, is a schematic representation of an event monitoring system 600. System 600 may be any of the systems described or otherwise envisioned herein, and may comprise any of the components described or otherwise envisioned herein.

According to an embodiment, system 600 comprises one or more of a processor 620, memory 630, user interface 640, communications interface 650, and storage 660, interconnected via one or more system buses 612. It will be understood that FIG. 6 constitutes, in some respects, an abstraction and that the actual organization of the components of the system 600 may be different and more complex than illustrated.

According to an embodiment, system 600 comprises a processor 620 capable of executing instructions stored in memory 630 or storage 660 or otherwise processing data to, for example, perform one or more steps of the method. Processor 620 may be formed of one or multiple modules. Processor 620 may take any suitable form, including but not limited to a microprocessor, microcontroller, multiple microcontrollers, circuitry, field programmable gate array (FPGA), application-specific integrated circuit (ASIC), a single processor, or plural processors.

Memory 630 can take any suitable form, including a non-volatile memory and/or RAM. The memory 630 may include various memories such as, for example L1, L2, or L3 cache or system memory. As such, the memory 630 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 600. It will be apparent that, in embodiments where the processor implements one or more of the functions described herein in hardware, the software described as corresponding to such functionality in other embodiments may be omitted.

User interface 640 may include one or more devices for enabling communication with a user. The user interface can be any device or system that allows information to be conveyed and/or received, and may include a display, a mouse, and/or a keyboard for receiving user commands. In some embodiments, user interface 640 may include a command line interface or graphical user interface that may be presented to a remote terminal via communication interface 650. The user interface may be located with one or more other components of the system, or may located remote from the system and in communication via a wired and/or wireless communications network.

Communication interface 650 may include one or more devices for enabling communication with other hardware devices. For example, communication interface 650 may include a network interface card (NIC) configured to communicate according to the Ethernet protocol. Additionally, communication interface 650 may implement a TCP/IP stack for communication according to the TCP/IP protocols. Various alternative or additional hardware or configurations for communication interface 650 will be apparent.

Storage 660 may include one or more machine-readable storage media such as read-only memory (ROM), random-access memory (RAM), magnetic disk storage media, optical storage media, flash-memory devices, or similar storage media. In various embodiments, storage 660 may store instructions for execution by processor 620 or data upon which processor 620 may operate. For example, storage 660 may store an operating system 661 for controlling various operations of system 600.

It will be apparent that various information described as stored in storage 660 may be additionally or alternatively stored in memory 630. In this respect, memory 630 may also be considered to constitute a storage device and storage 660 may be considered a memory. Various other arrangements will be apparent. Further, memory 630 and storage 660 may both be considered to be non-transitory machine-readable media. As used herein, the term non-transitory will be understood to exclude transitory signals but to include all forms of storage, including both volatile and non-volatile memories.

While system 600 is shown as including one of each described component, the various components may be duplicated in various embodiments. For example, processor 620 may include multiple microprocessors that are configured to independently execute the methods described herein or are configured to perform steps or subroutines of the methods described herein such that the multiple processors cooperate to achieve the functionality described herein. Further, where one or more components of system 600 is implemented in a cloud computing system, the various hardware components may belong to separate physical systems. For example, processor 620 may include a first processor in a first server and a second processor in a second server. Many other variations and configurations are possible.

According to an embodiment, system 600 may comprise or be in remote or local communication with a database or data source 615. Database 615 may be a single database or data source or multiple. Database 615 may comprise the input data which may be used to train the classifier, as described and/or envisioned herein.

According to an embodiment, storage 660 of system 600 may store one or more algorithms and/or instructions to carry out one or more functions or steps of the methods described or otherwise envisioned herein. For example, the system may comprise one or more of data processing instructions 662, training instructions 663, deviation detection module 664, abnormal value detection module 665, and/or reporting instructions 667.

According to an embodiment, data processing instructions 662 direct the system to retrieve and process input data which is used to either: (i) train the classifier 664/665 using the training instructions 663, or (ii) to perform analysis for the patient using the trained classifier 664/665. The data processing instructions 662 direct the system to, for example, receive or retrieve input data or medical data to be used by the system as needed, such as from database 615 among many other possible sources. As described above, the input data can comprise a wide variety of input types from a wide variety of sources.

According to an embodiment, the data processing instructions 662 also direct the system to process the input data to generate a plurality of features related to medical information for a plurality of patients, which are used to train the classifier. This can be accomplished by a variety of embodiments for feature identification, extraction, and/or processing. The outcome of the feature processing is a set of features related to telemetry monitoring for a cohort of previously monitored patients, which thus comprises a training data set that can be utilized to train the classifier.

According to an embodiment, training instructions 663 direct the system to utilize the processed data to train the classifier 664/665. The classifier can be any machine learning classifier sufficient to utilize the type of input data provided. Thus, the system comprises a trained classifier configured to determine a prediction of the patient's likelihood of being in or developing a clinical state.

According to an embodiment, reporting instructions 667 direct the system to generate and provide a report indicating a prediction of the patient's likelihood of being in or developing a clinical state. The reporting instructions 667 also direct the system to display the report on a display of the system. The display may comprise information about the patient, the parameters, the input data for the patient, and/or the patient's likelihood of being in or developing a clinical state. Other information is possible. Alternatively, the report may be communicated by wired and/or wireless communication to another device. For example, the system may communicate the report to a mobile phone, computer, laptop, wearable device, and/or any other device configured to allow display and/or other communication of the report.

### Example 1

Discussed below are two examples of how the event monitoring system might be utilized. It should be appreciated that these examples are non-limiting.

Some patient monitors have an event surveillance monitoring function, where clinicians can set deviation and abnormal value detection to monitor patients in real-time. This functionality has not been widely used due to a lack of temporal trend definition for different clinical conditions. Typically, to come up with quantitative temporal trend definitions, as one would need to gather a group of clinical opinion leaders and come up with consensus criteria. This process is costly and time consuming, and the consensus definition lacks validation using clinical data. With the event monitoring system described or otherwise envisioned herein, one would be able to automatically discover and validate temporal trend criteria at the same time. This would enable the event surveillance monitoring function to easily add pre-settings for different patient conditions. The system is also a tool that hospitals can use to find temporal trend definitions on data from their own hospital and apply those hospital-specific temporal criteria to their monitors.

For demonstration, the event monitoring system described or otherwise envisioned herein was utilized to discover vital trends for predicting shock. There was a dataset where hemodynamic interventions indicating possible shock had been labeled. Hemodynamically unstable patients comprise a small percentage of all patients (around 10%), which makes detecting unstable patients with high recall difficult. The event monitoring system was utilized to identify temporal patterns in three vital signs that are closely monitored for hemodynamic instability. The discovered temporal trend parameters were:

```
heartRate:
  greater than 161.0 for at least 17.0 min
  OR, 41.0 decrease in 15.0 mins
SpO2:
  less than 85.0 for at least 36.0 min
  OR, 22.0 decrease in 17.0 mins
systemicMean:
  less than 65.0 for at least 6.0 min
  OR, 56.0 decrease in 1.0 mins
```

These temporal trends can be directly applied to a ‘shock detection’ functionality on an event monitoring platform. Clinicians would simply turn on the ‘shock detection’ Advanced Event Surveillance and be alerted when their patient match the temporal event.

Using the temporal trends discovered here, one can detect hemodynamically unstable patients with a recall of 0.46 and a PPV of 0.60. This means that 46% of unstable patients are found while making fewer false positive predictions at a 40% false positive rate, equivalent to 60% PPV, which is a very high PPV compared to machine-learning methods that typically return more false positives and catch less true event cases for unbalanced datasets like this one.

### Example 2

The event monitoring system can also be used to improve the reliability and reduce false positive rate of predictive clinical scores by using temporal trends of clinical scores rather than hard triggers. Predictive clinical scores integrate information from multiple data sources, often including high-frequency temporal data. This exposes the scores to jumpiness and volatility. Typically, clinicians are alerted when a score threshold indicating great enough risk is crossed just once. Temporally noisy scores can have erroneous threshold crossings that lead to false alerts.

It has been that shown not alerting immediately when a threshold is crossed, but rather waiting some time period for the score to stay in the threshold region, leads to better predictive power. The question remains how to set the time period required for an alert. The event monitoring system allows automatic discovery of the optimal wait period along alongside the optimal score threshold using the batch abnormal detection module.

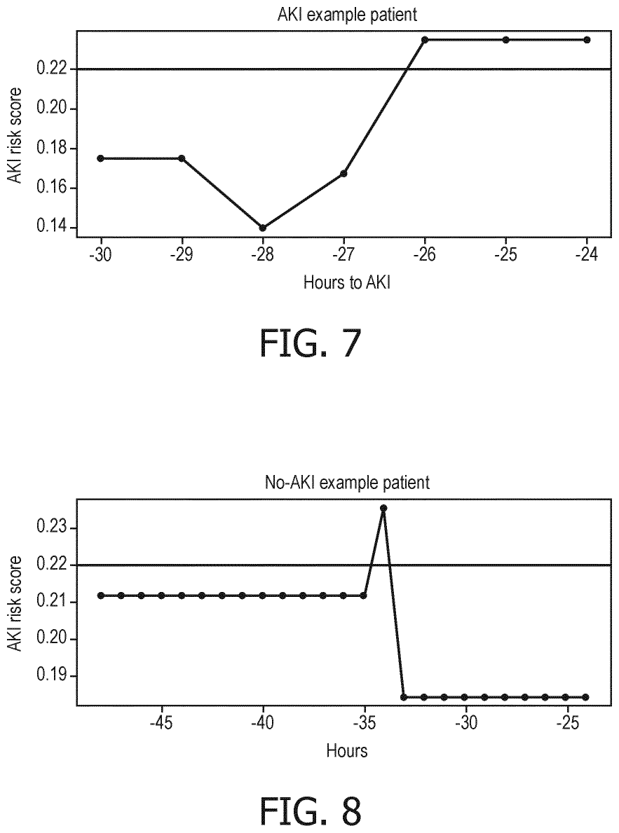

FIGS. 7 and 8 below provide examples of an acute kidney injury (AKI) risk score for predicting AKI using patient data. When the alerting threshold is set to a hard value of 0.22, some no-AKI (patients who never develop AKI, second figure) are predicted to be at high AKI risk due to one jumpy score. Using a dynamic alerting threshold criteria with an additional ‘time above threshold’ requirement will eliminate this patient example as false positive. Meanwhile, true-AKI patients (top example) satisfy the ‘time above threshold’ criteria and remain as true positives. The optimal threshold and ‘time above threshold’ can be found automatically from the data using the batch abnormal value detection module described in this invention. This method can be applied to any clinical score that changes rapidly in time.

### Example 3

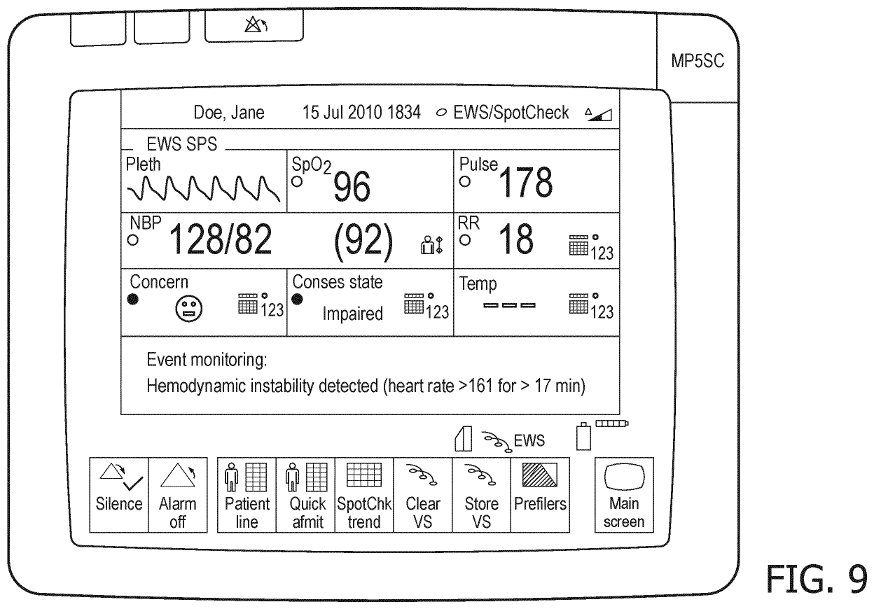

According to an embodiment, the event monitoring system can be incorporated into patient monitors such as the Philips® Intellivue® monitors, or other monitors. Referring to FIG. 9, for example, is a non-limiting example of the event monitoring system utilized in a patient monitor, which also monitors many other features of the patient. The event monitoring system has analyzed many time points of data for the patient, and determined that the heart rate has been greater than 161 bmp for more than 17 minutes, which triggers a warning that the patient is likely experience or susceptible to hemodynamic instability. Accordingly, the system has provided an alarm on the screen, which may be accompanied by an audible or other alarm or alert.

According to an embodiment, the event monitoring system is configured to process many thousands or millions of datapoints in the input data used to train the classifier, as well as in the received medical information utilized for decisions for a plurality of patients. For example, generating a functional and skilled trained classifier using an automated process such as feature identification and extraction and subsequent training requires processing of millions of datapoints from input data and the generated features. This can require millions or billions of calculations to generate a novel trained classifier from those millions of datapoints and millions or billions of calculations. As a result, each trained classifier is novel and distinct based on the input data and parameters of the machine learning algorithm. Thus, generating a functional and skilled trained classifier comprises a process with a volume of calculation and analysis that a human brain cannot accomplish in a lifetime, or multiple lifetimes.

Similarly, the event monitoring system can be configured to continually receive data about a patient, perform the analysis, and provide periodic or continual updates via the report for each patient. This requires the analysis of thousands or millions of datapoints on a continual basis to optimize the reporting, requiring a volume of calculation and analysis that a human brain cannot accomplish in a lifetime.

By providing a quick and thorough patient analysis, this novel event monitoring system has an enormous positive effect compared to prior art systems. As just one example, by providing a system that can factor in the best possible likelihood of a patient being in or developing a clinical state, the system will improve the survival outcomes and will lead to saved lives.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures.

1. A method for automated prediction of a clinical state using an event monitoring system, comprising:
receiving a set of parameter definitions for a clinical state, the set of parameter definitions comprising a definition for a deviation value, a definition for a deviation time, a definition for a value threshold, and a definition for a value time;
receiving a plurality of measurements for at least one feature for a patient, the plurality of measurements taken over a span of time;
identifying, within the plurality of measurements for at least one feature for a patient, a deviation and/or an abnormality predicting the clinical state, comprising:
comparing, using a batch deviation detection module of the event monitoring system, the received plurality of measurements for the patient to the defined deviation value and the defined deviation time; and
identifying, based on the comparing step, a deviation if the received plurality of measurements taken over the span of time for the patient is above the defined deviation value and below the defined deviation time; and/or
comparing, using the batch deviation detection module of the event monitoring system, the received plurality of measurements for the patient to the defined value threshold and the defined value time; and
identifying, based on the comparing step, an abnormality if the received plurality of measurements taken over the span of time for the patient is above the defined value threshold and below the defined value time;
predicting, based upon identification of a deviation and/or abnormality, that the patient is susceptible to or experiencing the clinical state;
providing, via a user interface of the event monitoring system, an alert that the patient is susceptible to or experiencing the clinical state.

2. The method of claim 1, wherein the plurality of measurements comprise measurements for a plurality of patients, and the method further comprises the step of organizing the plurality of measurements with individual patients within the plurality of patients.

3. The method of claim 1, further comprising the step of extracting one or more features from the received plurality of measurements.

4. The method of claim 1, wherein the feature is a vital sign, a lab result, and/or a clinical score.

5. The method of claim 1, further comprising the steps of: (i) receiving training data comprising medical information and clinical state information for a plurality of patients; (ii) extracting a plurality of features from the received training data; and (iii) training the batch deviation detection module.

6. The method of claim 1, wherein the event monitoring system comprises a clinical care system configured to monitor multiple life signs for a patient, and wherein the user interface of the event monitoring system comprises a display of the monitored multiple life signs for the patient, and wherein the alert that the patient is susceptible to or experiencing the clinical state comprises a textual alert on the display.

7. The method of claim 1, wherein the event monitoring system is utilized to determine a set of parameter definitions for a clinical state during training of the event monitoring system.

8. An event monitoring system, comprising:
a set of parameters definitions for a clinical state, the set of parameter definitions comprising a definition for a deviation value, a definition for a deviation time, a definition for a value threshold, and a definition for a value time;
a plurality of measurements for at least one feature for a patient, the plurality of measurements taken over a span of time;
a trained batch deviation detection module;
a processor configured to identify, within the plurality of measurements for at least one feature for a patient, a deviation and/or an abnormality predicting the clinical state, comprising: (i) comparing, using a batch deviation detection module of the event monitoring system, the received plurality of measurements for the patient to the defined deviation value and the defined deviation time; (ii) identifying, based on the comparing step, a deviation if the received plurality of measurements taken over the span of time for the patient is above the defined deviation value and below the defined deviation time; (iii) comparing, using the batch deviation detection module of the event monitoring system, the received plurality of measurements for the patient to the defined value threshold and the defined value time; and (iv) identifying, based on the comparing step, an abnormality if the received plurality of measurements taken over the span of time for the patient is above the defined value threshold and below the defined value time; the processor is further configured to predict, based upon identification of a deviation and/or abnormality, that the patient is susceptible to or experiencing the clinical state; and
a user interface configured to provide an alert that the patient is susceptible to or experiencing the clinical state.

9. The system of claim 8, wherein the plurality of measurements comprise measurements for a plurality of patients, and wherein the processor is further configured to organize the plurality of measurements with individual patients within the plurality of patients.

10. The system of any of claim 8, wherein the processor is further configured to extract one or more features from the received plurality of measurements.

11. The system of any of claim 8, wherein the feature is a vital sign, a lab result, and/or a clinical score.

12. The system of any of claim 8, wherein the processor is further configured to receive training data comprising medical information and clinical state information for a plurality of patients; extract a plurality of features from the received training data; and train the batch deviation detection module.

13. The system of any of claim 8, wherein the event monitoring system is a clinical care system configured to monitor multiple life signs for a patient, and wherein the user interface of the event monitoring system comprises a display of the monitored multiple life signs for the patient, and wherein the alert that the patient is susceptible to or experiencing the clinical state comprises a textual alert on the display.

14. The system of any of claim 8, wherein the event monitoring system is utilized to determine a set of parameter definitions for a clinical state during training of the event monitoring system.
